(19) United States
(12) Patent Application Publication
(10) Pub. No.: US 2007/0066518 A1
(43) Pub. Date: Mar. 22, 2007

(54) Pharmaceutical composition for inducing apoptosis comprising a fusion protein between bfl-1 and green fluorescent protein or a gene encoding same

(75) Inventors: Chul-Woo Kim, Kyungki-do (KR); Jae-Kyun Ko, Kyungki-do (KR); Wan-Seok Yang, Seoul (KR)

Correspondence Address: SUGHRUE MION, PLLC, 2100 PENNSYLVANIA AVENUE, N.W., SUITE 800, WASHINGTON, DC, 20037, US

(73) Assignees: Chul-Woo Kim, Kyungki-do (KR); NOUVAX CO., LTD., Seoul (KR)

(21) Appl. No.: 10/558,296

(22) PCT Filed: Jun. 3, 2004
(86) PCT No.: PCT/KR04/01324

§ 371 Date: Nov. 28, 2005

(30) Foreign Application Priority Data
Jun. 16, 2003 (KR) 10-2003-0038670

(51) Int. Cl. A61K 38/17; C07K 14/435

(52) U.S. Cl. 514/12; 530/350

(57) ABSTRACT

A pharmaceutical composition comprising a fusion protein between green fluorescent protein and Bfl-1 protein or a gene encoding the fusion protein, as well as a pharmaceutically acceptable carrier, is capable of efficiently inducing apoptosis and useful for treating cancer or cell hyperplasia.

### FIELD OF THE INVENTION

The present invention relates to a pharmaceutical composition for inducing apoptosis comprising a fusion protein between anti-apoptotic protein Bfl-1 and green fluorescent protein (GFP) or a gene encoding the fusion protein, as well as a pharmaceutically acceptable carrier.

### BACKGROUND OF THE INVENTION

It is widely known that Bcl-2 family proteins, which are characterized by their Bcl-2 homology (BH) domains, are key regulators of apoptosis (Adams J M, et al., Science 281(5381): 1322-1326, 1998).

Bcl-2 family proteins can be categorized into two groups depending on whether they promote or inhibit cell apoptosis: pro-apoptotic proteins (Bax, Bak, Bok, Bcl-Xs, Bid, Bad, Bik, etc.) and anti-apoptotic proteins (Bcl-2, Bcl-xL, Bfl-1, Bcl-w, Mcl-1, E1B-19K, Ced-9, etc.).

Bfl-1 has been known to have anti-apoptotic activity against various apoptotic signals. In particular, Bfl-1 suppresses staurosporine-induced apoptosis in Reh human B-lymphoblastic cells and in Molt-4 human T-leukemia cells (Ko J K, et al., Oncogene 22(16): 2457-2465, 2003; and Shim Y H, et al., Int. J. Hematol. 72(4): 484-490, 2000). The known anti-apoptotic mechanism of Bfl-1 when induced by staurosporine is that it inhibits the cleavage of Bid, caspases 3, 8 and 9 while preventing the loss of mitochondrial transmembrane potential (ΔΨₘ) (Shim Y H, et al., Int. J. Hematol. 72(4): 484-490, 2000).

However, it has been demonstrated that various proteases cleave several Bcl-2 family proteins, including Bcl-2, Bcl-xL, Bax and Bid, producing large C-terminal fragments with potent pro-apoptotic activity (Heng E H, et al., Science 278(5345): 1966-1968, 1997; and Clem R J, et al., Proc. Natl. Acad. Sci. USA 95: 554-559, 1998). For example, activated caspase 8 cleaves cytosolic Bid, which is in an inactive form, generating an active pro-apoptotic C-terminal fragment (tBid) that translocates to the mitochondria, stimulates the release of cytochrome c, and activates other pro-apoptotic proteins such as Bax and Bak. Anti-apoptotic members Bcl-2 and Bcl-xL are also cleaved by caspase 3 under apoptotic situations. Interestingly, cleavage of these proteins is a critical step for converting them from anti-apoptotic proteins to Bax-like pro-apoptotic proteins that further activate downstream caspases and promote the release of cytochrome c. Therefore, post-translational modification of Bcl-2 family proteins might be a crucial regulation mechanism of the Bcl-2 family proteins.

A recent publication disclosed that three-dimensional structures of anti-apoptotic and pro-apoptotic proteins, such as Bcl-xL, Bcl-2, Bax and Bid, are strikingly similar (Schendel S L, et al., Cell Death Differ. 5(5): 372-80, 1998). However, despite their structural similarity, it is still unclear how they regulate cellular apoptosis in opposite ways.

The present inventors have unexpectedly found that Bfl-1, an anti-apoptotic Bcl-2 family protein, is converted into a potent pro-apoptotic protein, also displaying anti-cancer activity, when fused to GFP. Based on the discovery, a pharmaceutical composition comprising a fusion protein between Bfl-1 and GFP or a gene encoding the fusion protein has been developed for use in treating cancer and cell hyperplasia.

### SUMMARY OF THE INVENTION

Accordingly, it is an object of the present invention to provide a pharmaceutical composition for inducing apoptosis comprising a fusion protein between GFP and Bfl-1 or a gene encoding the fusion protein, as well as a pharmaceutically acceptable carrier.

It is another object of the present invention to provide an anti-cancer agent comprising a fusion protein between Bfl-1 and GFP or a gene encoding the fusion protein.

### BRIEF DESCRIPTION OF THE DRAWINGS

The relevant objects and features of the present invention will become apparent from the following description of the invention, when taken in conjunction with the accompanying drawings, in which:

FIG. 1 shows fluorescent microscope photographs of HEK 293T cells at 24 hours after transfection with (a) GFP and Bcl-xL expression vectors; (b) GFP and Bfl-1 expression vectors; (c) GFP-Bcl-xL expression vector; and (d) GFP-Bfl-1 expression vector, respectively;

FIG. 2 is a photograph of agarose gel electrophoresis of cell extracts obtained from HEK 293T cells 48 hours after transfection with GFP and Bfl-1 expression vectors; GFP-Bcl-xL expression vector; and GFP-Bfl-1 expression vector, respectively;

FIG. 3 presents a diagram representing apoptosis levels versus time after transfection of HEK 293T cells with GFP expression vector; Bfl-1 and GFP expression vectors; Bcl-xL and GFP expression vectors; GFP-Bcl-xL expression vector; and GFP-Bfl-1 expression vector, respectively;

FIG. 4 provides a diagram comparing apoptosis levels induced in HEK 293T cells transfected with GFP expression vector; GFP-Bfl-1 expression vector where GFP is fused to the N-terminal of Bfl-1; and Bfl-1-GFP expression vector where GFP is fused to the C-terminal of Bfl-1, respectively;

FIG. 5 offers a diagram comparing apoptosis levels induced in HEK 293T cells transfected with GFP expression vector; GFP-Bfl-1 expression vector where GFP is fused to the N-terminal of Bfl-1; β-galactosidase expression vector; and GaIF-Bfl-1 expression vector where β-galactosidase is fused to the N-terminal of Bfl-1, respectively;

FIG. 6 illustrates the structures of deletion mutants where a part of Bfl-1 domains is removed from GFP-Bfl-1 fusion protein and that of GFP-Bfl-1 where β-galactosidase is fused to the N-terminal of Bfl-1;

FIG. 7 depicts a diagram comparing apoptosis levels induced in HEK 293T cells transfected with expression vectors of GFP, GFP-Bcl-xL, GFP-Bax, GFP-Bfl-1 and deletion mutants thereof, respectively; and

FIG. 8 explains a result of agarose gel electrophoresis of cell extracts obtained from HEK 293T cells 48 hours after transfection with GFP, GFP-Bcl-xL, GFP-Bax, GFP-Bfl-1 and GFP-BC expression vectors, respectively.

### DETAILED DESCRIPTION OF THE INVENTION

Human Bfl-1 is an anti-apoptotic Bcl-2 family member encoded by a gene having a 737 base pair nucleotide sequence of SEQ ID NO: 1 (GenBank Accession No: U27467). Bfl-1 protein encoded by the gene of SEQ ID NO: 1 has 175 amino acids in accordance with the amino acid sequence of SEQ ID NO: 2, and includes BH1 domain corresponding to amino acids ranging from 71 to 90 of SEQ ID NO: 2 and BH2 domain corresponding to amino acids ranging from 133 and 145 of SEQ ID NO: 2. Further, the C-terminal part of Bfl-1 corresponding to amino acids ranging from 147 to 175 of SEQ ID NO: 2 includes a transmembrane domain.

In the fusion protein of the present invention, Bfl-1 protein may be a full-length Bfl-1 protein of SEQ ID NO: 2 or a fragment thereof comprising amino acids ranging from 147 to 175 of SEQ ID NO: 2. Further, GFP of the fusion protein of the present invention may be fused to the N-terminal or the C-terminal of Bfl-1, preferably to the N-terminal.

GFP is a protein cloned from a jellyfish, Aquorea Victoria. GFP is one of the most widely used reporter proteins and produces a green light when illuminated with blue or UV light (Inouye S, et al., FEBS Letters 351(2): 211-14, 1994). The present invention employs a commercially available GFP expression vector, preferably pEGFP-C1 (Clontech). The GFP protein encoded by pEGFP-C1 is a mutant of wild-type GFP, which has been modified to produce a stronger green light. Further, when a foreign gene is inserted into multiple cloning sites of pEGFP-C1 vector, the inserted foreign gene is expressed in the form of a fusion protein with GFP.

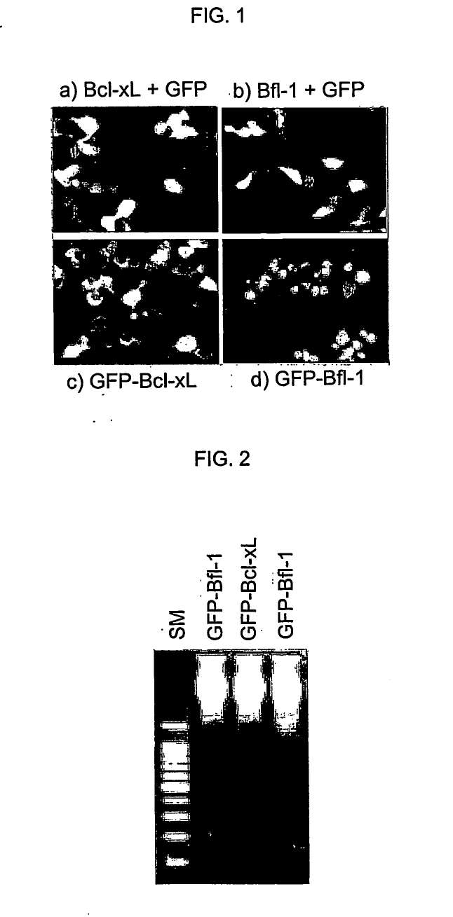

To investigate the fusion effect between GFP and Bfl-1 on anti-apoptotic activity of Bfl-1, HEK 293T cells were transfected with GFP and Bcl-xL expression vectors; GFP and Bfl-1 expression vectors; GFP-Bcl-xL expression vector where GFP is fused to the N-terminal of Bcl-xL; and GFP-Bfl-1 expression vector where GFP is fused to the N-terminal of Bfl-1, respectively. Thereafter, morphological features of the transfected cells were observed with a fluorescent microscope. Consequently, only HEK 293T cells transfected with GFP-Bfl-1 expression vector displayed the morphological features of apoptotic cells, namely, cell shrinkage, formation of cytoplasmic blebs and apoptotic bodies, and detachment from the culture plate, while Bfl-1 cotransfected together with GFP had no effect on cell death. In addition, other transient expression vectors, Bcl-xl-another anti-apoptotic Bcl-2 family member-together with GFP and GFP-Bcl-xL caused almost no pro-apoptotic effect on 293T cells (FIG. 1).

Further, cell extracts were obtained from the aforementioned transfected cells at 48 hours after transfection and subjected to agarose gel electrophoresis. As a result, chromosomal DNA fragmentation was observed in the cell extract of 293T cells transfected with GFP-Bfl-1 expression vector, demonstrating that the cell death was caused by apoptosis rather than necrosis (FIG. 2).

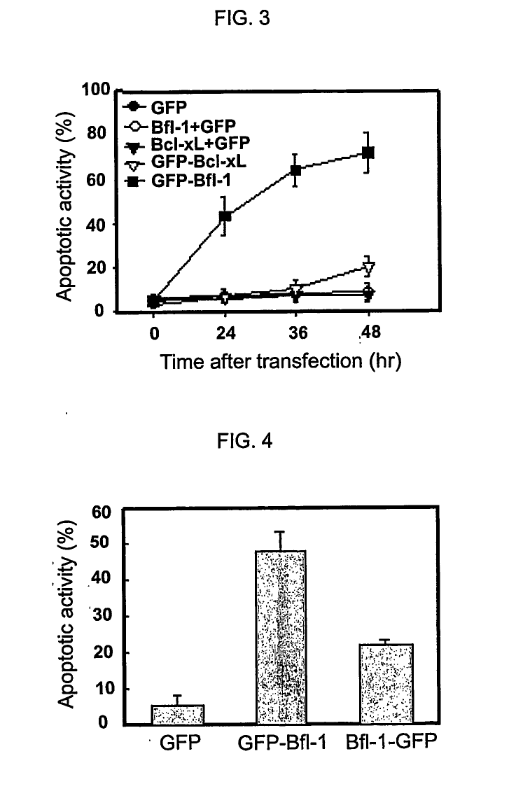

Moreover, when cell death levels were measured at 12, 24, 36, and 48 hours after transfection of HEK 293T cells with the aforementioned vectors, transient expression of GFP-Bfl-1 resulted in a marked apoptosis induction of 293T cells progressively with time. At 24 hours after transfection, apoptotic 293T cells transfected with GFP-Bfl-1 expression vector displayed greater than 50% death rate and greater than 90% at 48 hours after transfection. In contrast, transfection of other expression vectors had no significant effect on 293T cell death (FIG. 3). Since transient expression of GFP, GFP together with Bfl-1, or GFP-Bcl-xL fusion protein had no effect on cell death, the pro-apoptotic activity of GFP-Bfl-1 fusion protein did not result from GFP itself.

To investigate any effect of the fusion location of GFP on pro-apoptotic activity, GFP-Bfl-1 expression vector where GFP is fused to the N-terminal of Bfl-1 and Bfl-1-GFP expression vector where GFP is fused to the C-terminal of Bfl-1 were prepared and transfected into HEK 293T cells, respectively. When compared, GFP-Bfl-1 expression vector induced about 2.5-fold higher apoptotic activity than that of Bfl-1-GFP (FIG. 4). Accordingly, it is preferable to fuse GFP to the N-terminal of Bfl-1 to induce more effective cell apoptosis.

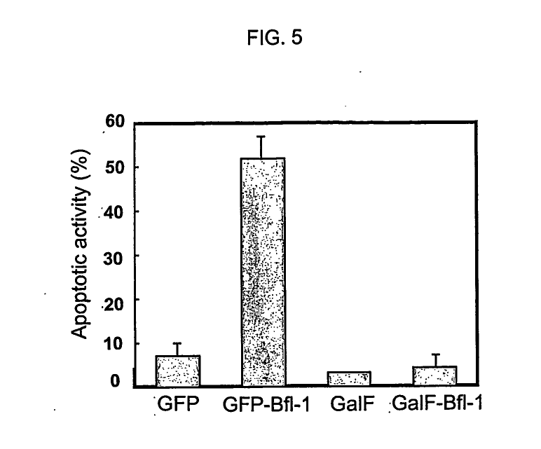

In addition, to investigate whether fusion between other reporter proteins and Bfl-1 would efficiently induce pro-apoptotic activity, a fragment of β-galactosidase (corresponding to amino acids ranging from 1 to 147) (MacGregor GR, et al., Nucleic Acids Res. 17:2365, 1989) was fused to the N-terminal of Bfl-1, to obtain GalF-Bfl-1 expression vector. When cell death levels were measured, it was confirmed that fusion with GFP induced 8-fold higher apoptosis than that with β-galactosidase (FIG. 5).

The above results demonstrate that the conversion of Bfl-1 to a pro-apoptotic protein is dependent on the fusion protein type (GFP vs. β-galactosidase) and partially dependent on the location of its fusion partner (N-terminal vs. C-terminal).

Meanwhile, Bfl-1 in the fusion protein of the present invention may be a deletion mutant, which lacks a part of Bfl-1 domains, such as N-terminal, BH1 and/or BH2 domains of Bfl-1.

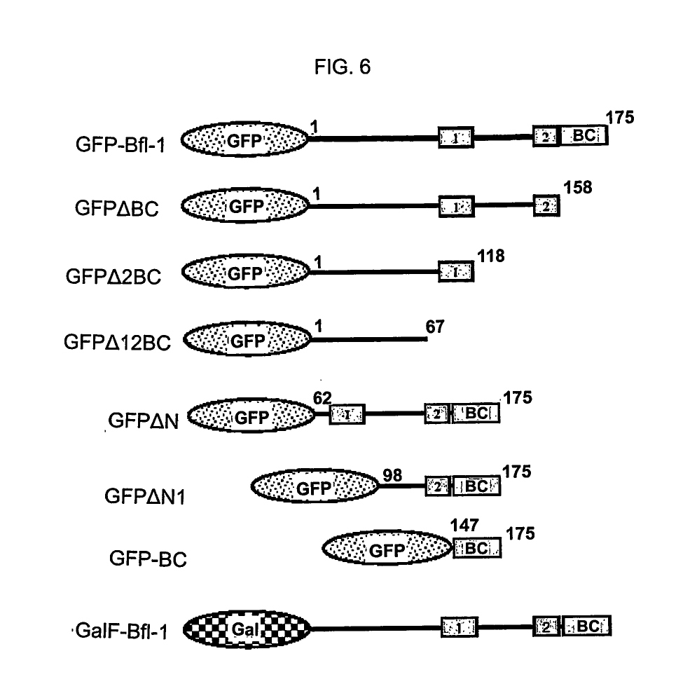

To investigate which domain of Bfl-1 selected from N-terminal, C-terminal, BH1 and BH2 domains was necessary for pro-apoptotic activity of the fusion protein, a series of GFP-Bfl-1 deletion mutants devoid of the aforementioned domains were prepared and transfected into HEK 293T cells. The specific deletion mutants were as follows: (1) GFPΔN lacking the N-terminal domain corresponding to the nucleotide sequence ranging from 1 to 61 of SEQ ID NO: 1; (2) GFPΔN1 lacking the N-terminal and BH1 domains corresponding to the nucleotide sequence ranging from 1 to 97 of SEQ ID NO: 1; (3) GFP-BC lacking the N-terminal, BH1, and BH2 domains corresponding to the nucleotide sequence ranging from 1 to 146 of SEQ ID NO: 1; (4) GFPΔBC lacking the C-terminal domain corresponding to the nucleotide sequence ranging from 159 to 175 of SEQ ID NO: 1; (5) GFPΔ2BC lacking the C-terminal and BH2 domains corresponding to the nucleotide sequence ranging from 119 to 175 of SEQ ID NO: 1; and (6) GFPΔ12BC lacking the C-terminal, BH1, and BH2 domains corresponding to the nucleotide sequence ranging from 68 to 175 of SEQ ID NO: 1 (FIG. 6).

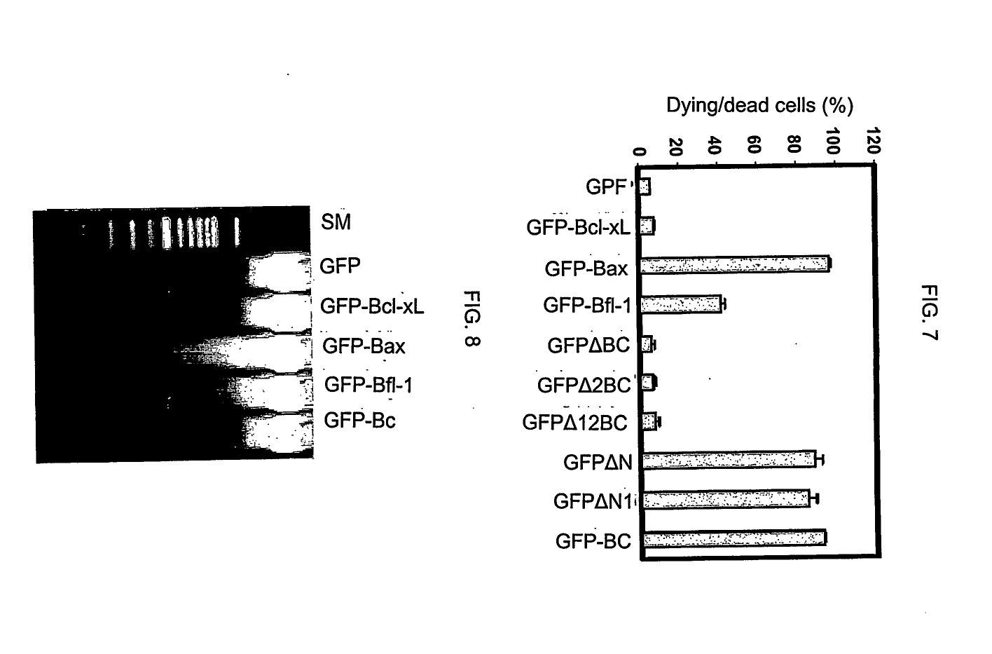

Each expression vector containing the above deletion mutants was transfected into BEK 293T cells according to the same method as described above, and the cell death levels were measured. As a result, the deletion mutants GFPΔBC, GFPΔ2BC, and GFPΔ12BC showed no pro-apoptotic effect on 293T cells, while the deletion mutants GFPΔN, GFPΔN1, and GFP-BC induced significantly higher apoptosis than GFP-Bfl-1. In particular, transient expression of GFP-BC lacking the N-terminal, BH1, and BH2 domains of Bfl-1 displayed over 90% apoptosis. Such strong pro-apoptotic activity of GFP-BC was far greater than that of GFP-Bax (Li X, et al., Cancer Res. 61(1): 186-191, 2001) (FIG. 7). These results suggest that the C-terminal domain of Bfl-1 is essential for the pro-apoptotic activity of GFP-Bfl-1 fusion protein.

Further, when cell extracts obtained from HEK 293T cells transfected with each deletion mutant were subjected to agarose gel electrophoresis, similar chromosomal DNA fragmentations were observed among the cells transfected with GFP-Bfl-1, GFP-BC, and GFP-Bax, demonstrating that the cell death was caused by apoptosis rather than necrosis (FIG. 8).

As described above, GFP-Bfl-1 fusion protein of the present invention strongly induced apoptotic cell death. In particular, GFP fusion at the N-terminal of Bfl-1 induced stronger pro-apoptotic activity than that at the C-terminal of Bfl-1. Further, the C-terminal domain of Bfl-1 was essential to induce the pro-apoptotic activity of GFP-Bfl-1 fusion protein.

A fusion protein between GFP and Bfl-1 or a gene encoding the fusion protein may be administered alone or in the form of a pharmaceutical composition together with pharmaceutically acceptable excipients, diluents, or carriers for apoptosis induction. In particular, a gene-delivering agent, preferably adenovirus, may be used to administer the gene that is inserted into the agent.

Adenovirus commonly used for the purpose of gene therapy has several advantages: it is easy to obtain high viral titer (about 10⁹ cfu/ml); the kinds of cells to be infected are not restricted; and its viral genome can express a target gene without integrating itself into a host chromosome.

In the pharmaceutical composition of the present invention, the gene encoding GFP-Bfl-1 fusion protein is selected from polynucleotides having the nucleotide sequences of SEQ ID NO: 23; SEQ ID NO: 24 lacking the N-terminal domain of Bfl-1; SEQ ID NO: 25 lacking the N-terminal and BH1 domains of Bfl-1; and SEQ ID NO: 26 lacking the N-terminal, BH1, and BH2 domains of Bfl-1.

Further, the pharmaceutical composition of the present invention may comprise a fusion protein selected from polypeptides having the amino acid sequences of SEQ ID NOs: 27 to 30 that are encoded by the nucleotide sequences of SEQ ID NOs: 23 to 26, respectively.

The pharmaceutical composition of the present invention may be pharmaceutically formulated in accordance with any one of the conventional procedures. In preparing the formulation, the effective ingredient is preferably admixed or diluted with a carrier, excipient, or diluent Examples of suitable carriers, excipients, or diluents are lactose, dextrose, sucrose, sorbitol, mannitol, glycine, polyethylene glycol, starches, gum acacia, alginates, gelatin, calcium phosphate, calcium silicate, cellulose, methylcellulose, microcrystalline cellulose, polyvinylpyrrolidone, water, methylhydroxybenzoates, propylhydroxybenzoates, talc, magnesium stearate, and mineral oil. The formulation may additionally include fillers, anti-agglutinating agents, lubricating agents, wetting agents, flavoring agents, emulsifiers, preservatives and the like. The composition of the invention may be formulated so as to provide a quick, sustained or delayed release of the active ingredient after it is administrated to a patient, by employing any one of the procedures well known in the art

The pharmaceutical composition of the present invention can be administered by injection (e.g., intramuscular, intravenous, intraperitoneal, subcutaneous), or by other methods such as infusion that ensure its delivery to the bloodstream in an effective form. The pharmaceutical composition may also be administered by intratumoral, peritumoral, intralesional or perilesional routes, to exert local as well as systemic therapeutic effects. Local or intravenous injection is preferred.

A typical daily dose of the fusion protein or gene encoding the fusion protein as an effective ingredient may range from about 0.01 to 100 mg/kg body weight, preferably 0.1 to 50 mg/kg body weight, and can be administered in a single or in multiple doses. However, it should be understood that the amount of the active ingredient actually administered may be determined in light of various relevant factors, including the condition to be treated; the chosen route of administration; the age, sex and body weight of the individual patient; and the severity of the patient's symptom. Therefore, the above doses are not intended to limit the scope of this invention in any way.

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usage and conditions.

### EXAMPLE 1

### Preparation of GFP Fusion Expression Vector

<1-1> Preparation of GFP-Bfl-1 and Bfl-1-GFP Expression Vectors

A total RNA was purified from a human blood sample (acquired from Seoul National University Hospital, Seoul, Korea) and subjected to RT-PCR using oligonucleotides of SEQ ID NOs: 3 and 4 as a primer set. RT-PCR was carried out using One-Step RT-PCR kit (Promega) according to the manufacture's instruction. The RT condition was one cycle of 45 minutes at 48° C., and the PCR condition was 40 cycles of 30 seconds at 94° C., 1 minute at 60° C., and 2 minutes at 68° C., and final extension of 7 minutes at 68° C. The amplified PCR product having an adenine (A) residue at both ends was cloned into TOPO 2.1 T vector (Invitrogen) without treating with a restriction enzyme. The resulting vector was digested with restriction enzymes EcoRI/BamHI to obtain a DNA fragment (570 base pairs) containing Bfl-1 gene, and then, the DNA fragment was cloned into pcDNA3.1myc/his(−)B vector (Invitrogen) pre-treated with the same restriction enzymes, to obtain Bfl-1 expression vector.

In order to amplify Bfl-1 gene, PCR was conducted using the oligonucleotides of SEQ ID NOs: 3 and 4 as a primer set and the Bfl-1 expression vector prepared above as a template. The PCR condition was 30 cycles of 1 minute at 94° C., 1 minute at 55° C, and 1 minute at 72° C. after initial denaturation of 5 minutes at 94° C., and final extension of 10 minutes at 72° C. The amplified PCR product was digested with restriction enzymes EcoRI/BamHI and subjected to agarose gel electrophoresis, to excise a DNA fragment of 525 base pairs in size. The DNA fragment was cloned into pEGFP-C1 and pEGFP-N1 vectors (Clontech) pre-treated with the same restriction enzymes, respectively, to obtain expression vectors Bfl-1-GFP where GFP was fused to the C-terminal of Bfl-1 and GFP-Bfl-1 where GFP was fused to the N-terminal of Bfl-1.

Sequencing analysis revealed that the expression vector GFP-Bfl-1 included a gene having the nucleotide sequence of SEQ ID NO: 23 wherein GFP was fused to the N-terminal of Bfl-1.

<1-2> Preparation of GFP-Bcl-xL Expression Vector

To construct an expression vector of GFP-Bcl-xL fusion protein, PCR was carried out using oligonucleotides of SEQ ID NOs: 5 and 6 as a primer set and pcDNA3-Bcl-xL expression vector containing Bcl-xL gene (acquired from Dr. Hong-Tae Kim of College of Medicine, the Catholic University of Korea) as a template. The PCR condition was 30 cycles of 1 minute at 94° C., 1 minute at 55° C., and 1 minute at 72° C. after initial denaturation of 5 minutes at 94° C., and final extension of 10 minutes at 72° C. The amplified PCR product was digested with restriction enzymes XhoI/EcoRI and subjected to agarose gel electrophoresis, to excise a DNA fragment of 700 base pairs in size. The DNA fragment was cloned into pEGFP-C1 vector (Clontech) pre-treated with the same restriction enzymes, to obtain GFP-Bcl-xL expression vector.

### EXAMPLE 2

### Pro-Apoptotic Activity of GFP-Bfl-1 Fusion Protein

Human embryonic kidney SEEK) 293T cells Korean Cell Line Bank), transformed with adenovirus proteins and SV40 large T antigen, were cultured at 37° C. for 24 hours in DMEM/F-12 medium (Life Technology) supplemented with 10% heat-inactivated fetal bovine serum, 100 U/ml penicillin, and 100 mg/ml streptomycin. For the morphological assessment of cell death, 293T cells were plated onto Lab Tek II Chamber Slides (Nalge Nunc International Naperville, Ill.) at the density of 5×10⁴ cells per well. After 24-hour incubation, 293T cells were transfected, using Lipofectamine procedure (Life Technology), with expression vectors Bfl-1, GFP-Bfl-1, and GFP-Bcl-xL, respectively.

Specifically, HEK 293T cells were transfected with GFP and Bcl-xL expression vectors; GFP and Bfl-1 expression vectors; GFP-Bcl-xL expression vector; and GFP-Bfl-1 expression vector, respectively (1 μg each). After 18-hour transfection, 293T cells were washed with PBS and fixed using 4% formaldehyde. The morphological features of 293T cells were observed using an Axiovert 100 inverted epifluorescence microscope (Carl Zeiss).

As shown in FIG. 1, at 12 hours after transfection of GFP-Bfl-1, a large number of 293T cells showed several morphologic features of apoptotic cells, such as cell shrinkage, formation of cytoplasmic blebs and apoptotic bodies, and detachment from a culture plate (FIG. 1(d)). However, transient expression of GFP together with Bcl-xL; GFP together with Bfl-1; and GFP-Bcl-xL had no pro-apoptotic effect on 293T cells (FIG. 1(a) to (c)). Accordingly, it was found that only the fusion protein between GFP and Bfl-1 showed pro-apoptotic activity.

For analysis of DNA fragmentation by agarose gel electrophoresis, 293T cells at 48 hours after transfection were scraped off and collected by centrifugation. The cell pellet was incubated with 0.2 mg/ml proteinase K (Gibco BRL) in 500 μl of a buffer solution (100 mM Tris-Cl, pH 8.5, 5 mM EDTA, 200 mM NaCl, 0.2% SDS) at 55° C. for 6 hours. The extracted DNA was precipitated with an equal volume of isopropanol. The precipitated DNA was treated with 0.1 mg/ml RNase A at 37° C., subjected to 2% agarose gel electrophoresis, and detected by ethidium bromide staining.

As shown in FIG. 2, the cells transfected with GFP-Bfl-1 showed chromatin condensation (under observation with fluorescence microscope) and chromosomal DNA fragmentation that are typical hallmarks of apoptotic cell death.

In addition, HEK 293T cells were transfected with expression vectors of GFP; GFP together with Bcl-xL; GFP together with Bfl-1; GFP-Bcl-xL; and GFP-Bfl-1, respectively. At 12, 24, 36, and 48 hours after transfection, 293T cells were washed with PBS and fixed with 4% formaldehyde. Subsequently, cells were stained with a solution containing 1 μg/ml of 4′6-diamidino-2-phenylindole (DAPI, Calbiochem) and visualized through the use of an Axiovert 100 inverted epifluorescence microscope (Carl Zeiss). Nuclei with rippled contours and chromatin condensation were considered to represent the apoptotic stage of 293T cells.

As shown in FIG. 3, transient expression of GFP-Bfl-1 resulted in a marked apoptotic activity induction of 293T cells in a time dependent manner. At 24 hours after transfection, apoptotic 293T cells were greater than 50% and about 90% of cells were dead by 48 hours after transfection, while transfection of other expression vectors, i.e., GFP; GFP and Bcl-xL; GFP and Bfl-1; and GFP-Bcl-xL, had no significant effect on 293T cell death.

### EXAMPLE 3

### Change in Pro-Apoptotic Activity According to GFP's Position to be Fused to Bfl-1

To investigate the effect of fusion location of GFP on pro-apoptotic activity, an expression vector of Bfl-1-GFP wherein GFP was fused to the C-terminal of Bfl-1 was constructed and its pro-apoptotic activity was compared with that of GFP-Bfl-1 expression vector prepared in Example 1.

HEK 293T cells were transfected with 1 g each of Bfl-1-GFP and GFP-Bfl-1 expression vectors, in accordance with Example 2.

At 12 hours after transfection, cells were washed with PBS and fixed using 4% formaldehyde. Subsequently, cells were stained with a solution containing 1 μg/ml of DAPI and visualized through the use of an Axiovert 100 inverted epifluorescence microscope (Carl Zeiss). In order to estimate the levels of cell death, the number of nuclei with rippled contours and chromatin condensation was counted. As a result, the fusion of GFP to the N-terminal of Bfl-1 showed about 2.5-fold higher pro-apoptotic activity than that to the C-terminal of Bfl-1 (FIG. 4).

These results suggested that the conversion of Bfl-1 to a pro-apoptotic protein was partially dependent on the location of fusion partner (N-terminal vs. C-terminal)

### EXAMPLE 4

### Change in Pro-Apoptotic Activity According to the Kind of Fusion Partner to be Fused to Bfl-1

To investigate whether Bfl-1 would show pro-apoptotic activity when fused with other fusion partners, a fusion protein between Bfl-1 and β-galactosidase (Gal) was constructed wherein Gal was fused to the N-terminal of Bfl-1.

In order to amplify Gal gene, PCR was carried out using oligonucleotides of SEQ ID NOs: 7 and 8 as a primer set and pCMV beta-Gal vector (Invitrogen) as a template, to amplify a β-galactosidase fragment (corresponding to amino acids ranging from 1 to 147) (MacGregor G R, et al., Nucleic Acids Res. 17:2365, 1989). The PCR condition was 30 cycles of 1 minute at 94° C., 1 minute at 55° C., and 1 minute at 72° C. after initial denaturation of 5 minutes at 94° C. and final extension of 10 minutes at 72° C. The amplified PCR product was digested with restriction enzymes XhoI/EcoRI and subjected to agarose gel electrophoresis, to excise a DNA fragment of 441 base pairs in size. The DNA fragment was cloned into pcDNA3.1myc/his(−)B expression vector pre-treated with the same restriction enzymes, to obtain GalF-Bfl-1 expression vector.

HEK 293T cells were transfected with 1.0 μg each of GFP; GFP-Bfl-1; GaIF; and GaIF-Bfl-1, in accordance with Example 2, and then, the induction levels of cell death were measured. As shown in FIG. 5, transient expression of GaIF-Bfl-1 showed significantly reduced pro-apoptotic effect on 293T cells, i.e., only 5% apoptosis induced by transfection with GFP-Bfl-1 expression vector.

These results suggested that the conversion of Bfl-1 to a pro-apoptotic protein depended on the kind of fusion partner (GFP vs. Gal).

### EXAMPLE 5

### Effects of Deletion of Bfl-1 Domains in GFP-Bfl-1 Fusion Protein on Pro-Apoptotic Activity

<5-1> Preparation of GFP-Bfl-1 Deletion Mutants Lacking a Part of Bfl-1 Domains

Since transient expression of GFP, GFP together Bfl-1, or GFP-Bcl-xL had no effect on cell death, the pro-apoptotic activity of GFP-Bfl-1 fusion protein did not result from GFP itself. However, there were several reports that GFP had cytotoxic effect on cells. Thus, it was necessary to investigate more thoroughly the role of Bfl-1 in the pro-apoptotic activity of GFP-Bfl-1 fusion protein.

The primary structure of Bfl-1 consists of conserved BH1 and BH2 domains and less conserved N-terminal and C-terminal domains. The present inventors serially deleted Bfl-1 domains in the direction from the N-terminal to the C-terminal in order to determine which region in Bfl-1 was important for GFP-Bfl-1-induced cell death.

Specifically, cDNA fragments of human Bfl-1 corresponding to amino acids ranging from 1 to 61, from 1 to 97, and from 1 to 146 were amplified by PCR using ExTaq. Polymerase (Takara). PCR was carried out using oligonucleotides of SEQ ID NOs: 9 and 10; SEQ ID NOs: 11 and 12; and SEQ ID NOs: 13 and 14, respectively, and Bfl-1 expression vector as a template. The PCR condition was 30 cycles of 1 minute at 94° C., 1 minute at 55° C., and 1 minute at 72° C. after initial denaturation of 5 minutes at 94° C., and final extension of 10 minutes at 72° C. The amplified PCR products were digested with restriction enzymes EcoRI/BamHI and subjected to agarose gel electrophoresis, to excise DNA fragments of 330, 231 and 90 base pairs in size, respectively.

The DNA fragments were cloned into pEGFP-C1 vector (Clontech), to prepare deletion mutant expression vectors GFP-ΔN (lacking the N-terminal domain), GFP-ΔN1 (lacking the N-terminal and BH1 domains) and GFP-BC (lacking the N-terminal, BH1, and BH2 domains), respectively (FIG. 6).

Further, cDNA fragments of human Bfl-1 corresponding to amino acids ranging from 159 to 175, from 119 to 175, and from 68 to 175 were amplified by PCR using ExTaq. Polymerase (Takara). PCR was carried out using oligonucleotides of SEQ ID NOs: 15 and 16; SEQ ID NOs: 17 and 18; and SEQ ID NOs: 19 and 20, respectively, and Bfl-1 expression vector as a template. The PCR condition was 30 cycles of 1 minute at 94° C., 1 minute at 55° C., and 1 minute at 72° C. after initial denaturation of 5 minutes at 94° C., and final extension of 10 minutes at 72° C. The amplified PCR products were digested with restriction enzymes EcoRI/XbaI and subjected to agarose gel electrophoresis, to excise DNA fragments of 474, 354 and 288 base pairs in size, respectively.

The DNA fragments were cloned into pEGFP-C1 vector (Clontech), to prepare deletion mutant expression vectors GFP-ΔBC (lacking the C-terminal domain), GFP-2ΔBC (lacking the C-terminal and BH2 domains) and GFP-12ΔBC (lacking the C-terminal, BH1, and BH2 domains), respectively (FIG. 6).

FIG. 6 shows the structures of deletion mutants lacking a part of Bfl-1 domains from GFP-Bfl-1 fusion protein, wherein 1, 2, and BC represent BH1 domain, BH2 domain, and the C-terminal domain of Bfl-1, respectively.

Meanwhile, a fusion protein between GFP and Bax, a pro-apoptotic protein, was prepared as a positive control as follows: to amplify Bax gene, PCR was carried out using oligonucleotides of SEQ ID NOs: 21 and 22 as a primer set and pcDNA3-Bax expression vector containing Bax gene (acquired from Dr. Hong-Tae Kim of College of Medicine, the Catholic University of Korea) as a template. The PCR condition was 30 cycles of 1 minute at 94° C., 1 minute at 55° C., and 1 minute at 72° C. after initial denaturation of 5 minutes at 94° C., and final extension of 10 minutes at 72° C. The amplified PCR product was digested with restriction enzymes Bgl II/HindIII and cloned into pEGFP-C1 vector (Clontech) pre-treated with the same restriction enzymes, to obtain GFP-Bax expression vector.

<5-2> Pro-Apoptotic Activity of GFP-Bfl-1 Deletion Mutants

HEK 293T cells were transfected with 1 μg each of GFPΔBC, GFPΔ2BC, GFPΔ12BC, GFPΔN, GFPΔN1, and GFP-BC deletion mutant expression vectors prepared in Example <5-1>; GFP-Bcl-xL expression vector; GFP-Bfl-1 expression vector; and GFP-Bax expression vector as a positive control and GFP expression vector as a negative control, respectively, in accordance with Example 2.

After 12-hour transfection, 293T cells were washed with PBS and fixed using 4% formaldehyde. Subsequently, cells were stained with a solution containing 1 μg/ml of DAPI (Calbiochem). In order to estimate the levels of cell death, the number of nuclei with rippled contours and chromatin condensation was counted.

At 24 hours after transfection, cell viability was monitored under a fluorescence microscope. As shown in FIG. 7, it was found that transient expression of GFP; GFP-Bcl-xL; GFP-Bfl-1; GFPΔBC; GFPΔ2BC; GFPΔ12BC; GFPΔN; GFPΔN1; GFP-BC; and GFP-Bax induced 5, 3, 45, 5, 6, 8, 91, 83, 93, and 85% apoptosis, respectively.

Accordingly, GFPΔN, GFPΔN1 and GFP-BC deletion mutants significantly enhanced pro-apoptotic activity compared to GFP-Bfl-1, while GFPΔBC, GFPΔ2BC and GFPΔ12BC deletion mutants showed no pro-apoptotic effect on 293T cells. These results suggested that GFP-Bfl-1 fusion proteins lacking the N-terminal, BH1 and/or BH2 domains of Bfl-1 can induce pro-apoptotic activity, but the C-terminal domain of Bfl-1 is essential for the pro-apoptotic activity of GFP-Bfl-1 fusion protein.

Further, each cell extract obtained from HEK 293T cells transfected with the deletion mutant expression vectors, was subjected to agarose gel electrophoresis in accordance with Example 2. As a result, transient expression of GFP-Bfl-1 and GFP-BC expression vectors only, showed similar chromosome fragmentation to that of GFP-Bax, demonstrating that the cell death was caused by apoptosis rather than necrosis.

Upon sequencing analysis of GFPΔN, GFPΔN1, and GFP-BC deletion mutants showing pro-apoptotic activity, it was revealed that they contained GFP-Bfl-1 deletion mutant genes having the nucleotide sequences of SEQ ID NOs: 24 to 26, respectively.

While the embodiments of the subject invention have been described and illustrated, it is obvious that various changes and modifications can be made therein without departing from the spirit of the present invention, which should be limited only by the scope of the appended claims.

What is claimed is:

1. A pharmaceutical composition for inducing apoptosis comprising a fusion protein and a pharmaceutically acceptable excipient, diluent, or carrier, wherein the fusion protein is prepared by fusing a green fluorescent protein to a Bfl-1 protein of SEQ ID NO: 2 or to a fragment of the Bfl-1 protein comprising amino acids ranging from 147 to 175 of SEQ ID NO: 2.

2. The pharmaceutical composition of claim 1, wherein the green fluorescent protein is fused to the N-terminal of the Bfl-1 protein or the fragment of the Bfl-1 protein.

3. The pharmaceutical composition of claim 1, wherein the fragment of the Bfl-1 protein is a polypeptide selected from the group consisting of amino acids ranging from 62 to 175 of SEQ ID NO: 2; amino acids ranging from 98 to 175 of SEQ ID NO: 2; and amino acids ranging from 147 to 175 of SEQ ID NO: 2.

4. The pharmaceutical composition of claim 1, wherein the fusion protein is a polypeptide selected from the group consisting of the polypeptides having the amino acid sequences of SEQ ID NOs: 27 to 30.

5. The pharmaceutical composition of claim 1, which is used for treating cancer or cell hyperplasia.

6. A pharmaceutical composition for inducing apoptosis comprising a gene encoding a fusion protein and a pharmaceutically acceptable excipient, diluent, or carrier, wherein the fusion protein is prepared by fusing a green fluorescent protein to a Bfl-1 protein of SEQ ID NO: 2 or a fragment of the Bfl-1 protein comprising amino acids ranging from 147 to 175 of SEQ ID NO: 2.

7. The pharmaceutical composition of claim 6, wherein the green fluorescent protein is fused to the N-terminal of the Bfl-1 protein or the fragment of the Bfl-1 protein.

8. The pharmaceutical composition of claim 6, wherein the fragment of the Bfl-1 protein is a polypeptide selected from the group consisting of amino acids ranging from 62 to 175 of SEQ ID NO: 2; amino acids ranging from 98 to 175 of SEQ ID NO: 2; and amino acids ranging from 147 to 175 of SEQ ID NO: 2.

9. The pharmaceutical composition of claim 6, wherein the fusion protein is a polypeptide selected from the group consisting of the polypeptides having the amino acid sequences of SEQ ID NOs: 27 to 30.

10. The pharmaceutical composition of claim 6, wherein the gene is selected from the group consisting of the polynucleotides having the nucleotide sequences of SEQ ID NOs: 23 to 26.

11. The pharmaceutical composition of claim 6, which is used for treating cancer or cell hyperplasia.


SEQUENCE LISTING (30 sequences)

SEQ ID NO: 1 - DNA, 737 bp, Homo sapiens Bfl-1
  CDS (35)..(559)
ccagctcaagactttgctctccaccaggcagaagatgacagactgtgaatttggatatatttacaggctggctcaggactatctgcagtgcgtcctacagataccacaacctggatcaggtccaagcaaaacgtccagagtgctacaaaatgttgcgttctcagtccaaaaagaagtggaaaagaatctgaagtcatgcttggacaatgttaatgttgtgtccgtagacactgccagaacactattcaaccaagtgatggaaaaggagtttgaagacggcatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttgaccagaaaggacactccatattgtgaaaccggcctaatttttctgactgatatggaaacgattgccaacacatacttctacttttaaataaacaactttgatgatgtaacttgaccttccagagttatggaaattttgtccccatgtaatgaataaattgtatgtatttttctcta

SEQ ID NO: 2 - protein, 175 aa, Homo sapiens Bfl-1
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 3 - DNA, 31 bp, Artificial Sequence
  forward primer specific for Bfl-1
gaattcgatgacagactgtgaatttggatat

SEQ ID NO: 4 - DNA, 30 bp, Artificial Sequence
  reverse primer specific for Bfl-1
ggatcctcaacagtattgcttcaggagaga

SEQ ID NO: 5 - DNA, 29 bp, Artificial Sequence
  forward primer specific for Bcl-xL
ctcgagaaatgtctcagagcaaccgggag

SEQ ID NO: 6 - DNA, 29 bp, Artificial Sequence
  reverse primer specific for Bcl-xL
gaattcggtcayttccgactgaagagtga

SEQ ID NO: 7 - DNA, 34 bp, Artificial Sequence
  forward primer specific for GalF
aactcgagatggatcccgtcgttttacaacgtcg

SEQ ID NO: 8 - DNA, 29 bp, Artificial Sequence
  reverse primer specific for GalF
aagaattccagatgaaacgccgagttaac

SEQ ID NO: 9 - DNA, 32 bp, Artificial Sequence
  forward primer specific for GFP-DelN
aagaattcgatggtgtccgtagacactgccag

SEQ ID NO: 10 - DNA, 30 bp, Artificial Sequence; identical to SEQ ID NO: 4
  reverse primer specific for GFP-DelN

SEQ ID NO: 11 - DNA, 31 bp, Artificial Sequence
  forward primer specific for GFP-DelN1
gaaggtattctcatcaagaaacttctacgac

SEQ ID NO: 12 - DNA, 30 bp, Artificial Sequence; identical to SEQ ID NO: 4
  reverse primer specific for GFP-DelN1

SEQ ID NO: 13 - DNA, 34 bp, Artificial Sequence
  forward primer specific for GFP-BC
gatcccgaattctaagtttgaacctaaatctggc

SEQ ID NO: 14 - DNA, 36 bp, Artificial Sequence
  reverse primer specific for GFP-BC
gatcccggatcctcaacagtattgcttcaggagaga

SEQ ID NO: 15 - DNA, 31 bp, Artificial Sequence; identical to SEQ ID NO: 3
  forward primer specific for GFP-DelBC

SEQ ID NO: 16 - DNA, 31 bp, Artificial Sequence
  reverse primer specific for GFP-DelBC
tctagaaaagtcatccagccagatttaggtt

SEQ ID NO: 17 - DNA, 31 bp, Artificial Sequence; identical to SEQ ID NO: 3
  forward primer specific for GFP-Del2BC

SEQ ID NO: 18 - DNA, 30 bp, Artificial Sequence
  reverse primer specific for GFP-Del2BC
aagaattcaaatctccttataggtatccac

SEQ ID NO: 19 - DNA, 31 bp, Artificial Sequence; identical to SEQ ID NO: 3
  forward primer specific for GFP-Del12BC

SEQ ID NO: 20 - DNA, 27 bp, Artificial Sequence
  reverse primer specific for GFP-Del12BC
aagaattcaggcagtgtctacggacac

SEQ ID NO: 21 - DNA, 36 bp, Artificial Sequence
  forward primer specific for Bax
gatcccaagctttcagcccatcttcttccagatggt

SEQ ID NO: 22 - DNA, 32 bp, Artificial Sequence
  reverse primer specific for Bax
gatcccagatctatggacgggtccggggagca

SEQ ID NO: 23 - DNA, 1281 bp, Artificial Sequence
  GFP-Bfl-1 fusion protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattcgatgacagactgtgaatttggatatatttacaggctggctcaggactatctgcagtgcgtcctacagataccacaacctggatcaggtccaagcaaaacgtccagagtgctacaaaatgttgcgttctcagtccaaaaagaagtggaaaagaatctgaagtcatgcttggacaatgttaatgttgtgtccgtagacactgccagaacactattcaaccaagtgatggaaaaggagtttgaagacggcatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttga

SEQ ID NO: 24 - DNA, 1101 bp, Artificial Sequence
  GFP-DelN fusion protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattcgatggtgtccgtagacactgccagaacactattcaaccaagtgatggaaaaggagtttgaagacggcatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttga

SEQ ID NO: 25 - DNA, 990 bp, Artificial Sequence
  GFP-DelN1 fusion protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattcgattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttga

SEQ ID NO: 26 - DNA, 843 bp, Artificial Sequence
  GFP-BC fusion protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattcgaagtttgaacctaaatctggctggatgacttttctagaagttacaggaaagatctgtgaaatgctatctctcctgaagcaatactgttga

SEQ ID NO: 27 - protein, 426 aa, Artificial Sequence [sequence omitted]

SEQ ID NO: 28 - protein, 366 aa, Artificial Sequence
  GFP-DelN fusion protein
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKSGLRSRAQASNSMVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 29 - protein, 329 aa, Artificial Sequence
  GFP-DelN1 fusion protein
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKSGLRSRAQASNSILIKKLLRQQIAPDVDTYKEISYFVAEFIMNNTGEWIRQNGGWENGFVKKFEPKSGWMTFLEVTGKICEMLSLLKQYC

SEQ ID NO: 30 - protein, 280 aa, Artificial Sequence
  GFP-BC fusion protein
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKSGLRSRAQASNSKFEPKSGWMTFLEVTGKICEMLSLLKQYC